(19) United States
(12) Patent Application Publication
(10) Pub. No.: US 2022/0297616 A1
(43) Pub. Date: Sep. 22, 2022

(54) STEERING WHEEL STERILIZATION SYSTEM AND METHOD

(71) Applicant: HYUNDAI MOBIS CO., LTD., Seoul (KR)

(72) Inventor: Chang Won LEE, Seoul (KR)

(73) Assignee: HYUNDAI MOBIS CO., LTD., Seoul (KR)

(21) Appl. No.: 17/693,862

(22) Filed: Mar. 14, 2022

(30) Foreign Application Priority Data
Mar. 17, 2021 (KR) 10-2021-0034875

(51) Int. Cl. B60R 15/00; B62D 1/06; A61L 2/24; A61L 2/10; B60H 3/00

(52) U.S. Cl. CPC B60R 15/00; B62D 1/065; A61L 2/24; A61L 2/10; B60H 3/0078; B62D 1/04

(57) ABSTRACT

Provided is a steering wheel sterilization system including a sterilizing device for sterilizing a steering wheel of a vehicle, a grip recognition unit for recognizing a position on the steering wheel gripped by a driver and/or a time for which the driver grips the steering wheel, and a controller for controlling operation of the sterilizing device on the basis of the position on the steering wheel gripped by the driver and/or the time for which the driver grips the steering wheel, recognized by the grip recognition unit.

### CROSS-REFERENCE TO RELATED APPLICATIONS

This application claims the benefit of Korean Patent Application No. 10-2021-0034875, filed on Mar. 17, 2021, in the Korean Intellectual Property Office, the disclosure of which is incorporated herein by reference.

### BACKGROUND

### 1. Technical Field

The present invention relates to a steering wheel sterilization system and method, and specifically, to a technique for sterilizing a steering wheel on the basis of a time for which a driver grips the steering wheel or a position on the steering wheel gripped by the driver.

### 2. Discussion of Related Art

In general, a steering wheel is a device that changes a traveling direction of a vehicle by a hand operation of a driver riding in the vehicle. Driver's hands are in contact with a steering wheel from starting of a vehicle until the vehicle arrives at a destination.

To sterilize such a steering wheel, conventionally, a steering wheel sterilizing device for sterilizing a steering wheel using ultraviolet rays has been developed.

However, such a steering wheel sterilizing device is operated only by an operation of a driver, and thus there is a need for a technique for controlling a periodic operation of a steering wheel sterilizing device.

The matters described as the background art above are only for improving the understanding of the background of the present invention and should not be taken as acknowledging that they correspond to the prior art already known to those skilled in the art.

### SUMMARY

Therefore, the present invention has been made in view of the above problems, and it is an object of the present invention to provide a technique for detecting a time for which a driver grips a steering wheel or a position on the steering wheel gripped by the driver and controlling an operation of a sterilizing device on the basis of the detected time or position.

In accordance with the present invention, the above and other objects can be accomplished by the provision of a steering wheel sterilization system including a sterilizing device for sterilizing a steering wheel of a vehicle, a grip recognition unit for recognizing a position on the steering wheel gripped by a driver and/or a time for which the driver grips the steering wheel, and a controller for controlling operation of the sterilizing device on the basis of the position on the steering wheel gripped by the driver and/or the time for which the driver grips the steering wheel, recognized by the grip recognition unit.

The grip recognition unit may be connected to a camera sensor provided in the vehicle and/or a pressure sensor mounted on the steering wheel and recognize a position on the steering wheel gripped by a driver and/or a time for which the driver grips the steering wheel.

The steering wheel sterilization system may further include a driver recognition unit for recognizing whether a driver is riding in the vehicle and/or information on the driver, wherein the controller may control the operation of the sterilizing device on the basis of whether a driver is riding in the vehicle or information on the driver recognized by the driver recognition unit.

The controller may control the sterilizing device to operate when the driver recognition unit recognizes that the driver has alighted from the vehicle.

The controller may simultaneously operate the sterilizing device and a heater of the vehicle.

The controller may store the position on the steering wheel gripped by the driver and/or the time for which the driver grips the steering wheel, recognized by the grip recognition unit, according to the information on the driver recognized by the driver recognition unit and control the sterilizing device such that the sterilizing device immediately operates depending on the stored position on the steering wheel gripped by the driver and/or the stored time for which the driver grips the steering wheel according to the information on the driver.

The driver recognition unit may be connected to a camera sensor provided in the vehicle and recognize whether a driver is riding in the vehicle and/or information on the driver.

The controller may control an operating range of the sterilizing device depending on the position on the steering wheel gripped by the driver, recognized by the grip recognition unit.

The controller may control an intensity of operation of the sterilizing device depending on the time for which the driver grips the steering wheel, recognized by the grip recognition unit.

The steering wheel sterilization system may further include a signal generator for generating a steering wheel sterilization request signal depending on the position on the steering wheel gripped by the driver and/or the time for which the driver grips the steering wheel, recognized by the grip recognition unit.

The signal generator may generate a steering wheel sterilization request signal through a display device and/or a speaker device provided in the vehicle.

A method for controlling the steering wheel sterilization system according to claim 1 includes recognizing a position on the steering wheel gripped by a driver and/or a time for which the driver grips the steering wheel, and controlling operation of a sterilizing device on the basis of the recognized position on the steering wheel gripped by the driver and/or the recognized time for which the driver grips the steering wheel.

The method may further include recognizing whether or not a driver is riding in a vehicle, wherein the controlling of the operation of the sterilizing device may include operating the sterilizing device upon recognizing that the driver has alighted from the vehicle.

The method may further include simultaneously operating a heater of the vehicle when the sterilizing device operates after the driver has alighted from the vehicle.

The method may further include recognizing information on the driver, and storing a position on the steering wheel gripped by a driver and/or a time for which the driver grips the steering wheel according to the information on the driver, wherein the controlling of the operation of the sterilizing device may include operating the sterilizing device depending on the stored position on the steering wheel gripped by the driver and/or the stored time for which the driver grips the steering wheel.

The method may further include generating a steering wheel sterilization request signal depending on the position on the steering wheel gripped by the driver and/or the time for which the driver grips the steering wheel, recognized in the recognizing of a position on the steering wheel gripped by a driver and/or a time for which the driver grips the steering wheel before the controlling of the operation of the sterilizing device.

The steering wheel sterilization system according to the present invention can maintain a steering wheel in a clean state by detecting a time for which a driver grips the steering wheel or a position on the steering wheel gripped by the driver and controlling the operation of the sterilizing device on the basis of the detected time or position.

In addition, it is possible to recognize whether a driver is riding in the vehicle and information on the driver, store a time for which a driver grips the steering wheel or a position on the steering wheel gripped by the driver, and control the sterilizing device such that it operates when the driver has alighted from the vehicle, thereby providing the steering wheel in a clean state when the driver rides in the vehicle.

Furthermore, it is possible to maximize the sterilization effect by simultaneously operating the sterilizing device and a heater of the vehicle.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram of a steering wheel sterilization system according to an embodiment of the present invention;

FIG. 2 is a diagram illustrating a state in which a sterilizing device according to an embodiment of the present invention is mounted on a steering wheel;

FIG. 3 is a diagram illustrating a state in which a camera sensor according to an embodiment of the present invention recognizes gripping of a steering wheel and a driver; and

FIG. 4 is a flowchart of a steering wheel sterilization method according to an embodiment of the present invention.

### DETAILED DESCRIPTION

Specific structural and functional descriptions of embodiments of the present invention disclosed in the present specification or application are illustrated for the purpose of describing embodiments according to the present invention, and embodiments according to the present invention may be implemented in various forms and should not be construed as being limited to the embodiments described in the present specification and application.

While embodiments according to the present invention are susceptible to various modifications and alternative forms, specific embodiments are shown by way of example in the drawings. However, the present invention should not be construed as limited to the embodiments set forth herein, but on the contrary, the present invention is to cover all modifications, equivalents, and alternatives falling within the spirit and scope of the present invention.

The terms “first” and/or “second” are used to describe various components, but such components are not limited by these terms. The terms are used to discriminate one component from another component. For example, a first component may be called a second component and the second component may be called the first component within the technical spirit of the present invention.

When a component is “coupled” or “connected” to another component, it should be understood that a third component may be present between the two components although the component may be directly coupled or connected to the other component. When a component is “directly coupled” or “directly connected” to another component, it should be understood that no element is present between the two components. Further, other representations describing a relationship between components, that is, “between,” “immediately between,” “adjacent to” and “directly adjacent to” should be construed likewise.

The terms used in the specification of the present invention are merely used in order to describe particular embodiments, and are not intended to limit the scope of the present invention. An element described in the singular form is intended to include a plurality of elements unless the context clearly indicates otherwise. In the specification of the present invention, it will be further understood that the term “comprise” or “include” specifies the presence of a stated feature, figure, step, operation, component, part or a combination thereof, but does not preclude the presence or addition of one or more other features, figures, steps, operations, components, or combinations thereof.

All the terms that are technical, scientific or otherwise agree with the meanings as understood by a person skilled in the art unless defined to the contrary. Common terms as found in dictionaries should be interpreted in the context of the related technical writings not too ideally or impractically unless expressly disclosed herein.

Hereinafter, preferred embodiments of the present invention will be described in detail with reference to the attached drawings. Throughout the drawings, the same reference numerals represent the same members.

A controller 30 according to an exemplary embodiment of the present invention may be implemented through a non-volatile memory (not shown) configured to store an algorithm configured to control operations of various components of a vehicle or data with respect to software instructions for reproducing the algorithm, and a processor (not shown) configured to perform operations which will be described below using data stored in the memory. Here, the memory and the processor may be implemented as separate chips. Alternatively, the memory and the processor may be implemented as a single integrated chip. The processor may take the form of one or more processors.

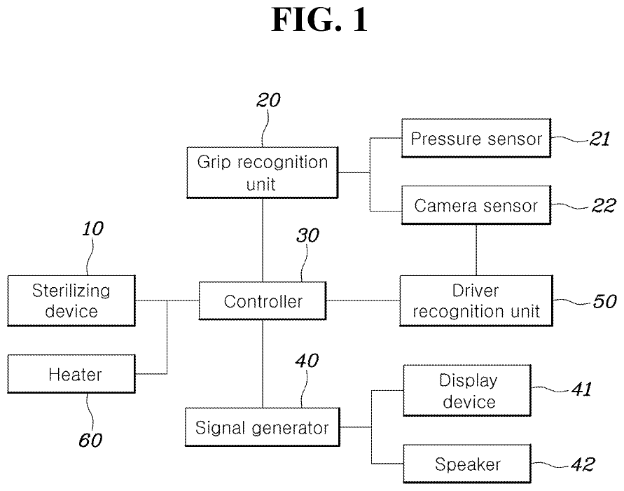

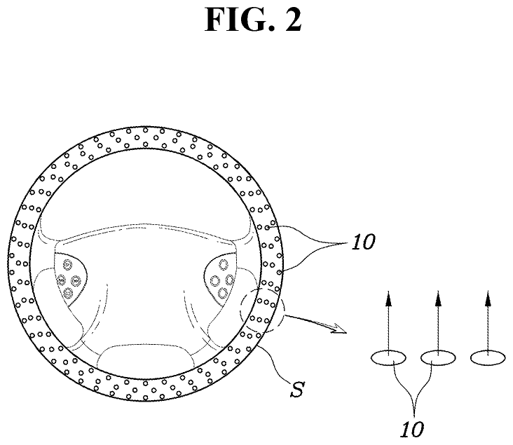

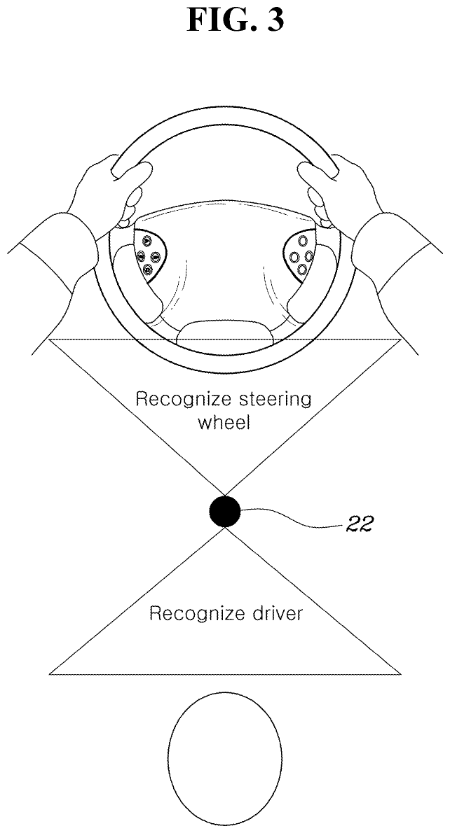

FIG. 1 is a block diagram of a steering wheel sterilization system according to an embodiment of the present invention, FIG. 2 is a diagram illustrating a state in which a sterilizing device 10 according to an embodiment of the present invention is mounted on a steering wheel, and FIG. 3 is a diagram illustrating a state in which a camera sensor 22 according to an embodiment of the present invention recognizes gripping of the steering wheel and a driver.

A preferred embodiment of the steering wheel sterilization system according to the present invention will be described with reference to FIGS. 1 to 3.

The steering wheel sterilization system according to the present invention includes the sterilizing device 10 for sterilizing a steering wheel, a grip recognition unit 20 for detecting or recognizing a grip position of a portion on the steering wheel gripped by a driver, or a grip time for which the driver grips the steering wheel, and the controller 30 for controlling the operation of the sterilizing device 10 on the basis of the position on the steering wheel gripped by the driver or the time for which the driver grips the steering wheel recognized by the grip recognition unit 20.

As shown in FIG. 1 to FIG. 3, the sterilizing device 10 includes an ultraviolet lamp that is provided on one side inside the steering wheel and emits ultraviolet rays through optical fibers disposed along the rim of the steering wheel, and the optical fibers mounted on the steering wheel with the outside thereof covered with a cover in which a plurality of discharge holes are formed.

Accordingly, the sterilizing device 10 can disinfect not only the steering wheel but also driver's hands during operation.

In addition, the sterilizing device 10 may be applied in various manners in addition to the above-described embodiment.

The grip recognition unit 20 may be connected to various sensors provided in the vehicle and calculate a position on the rim of the steering wheel gripped by the driver and a time for which the driver grips the rim of the steering wheel during driving of the vehicle.

The controller 30 may set an operating range of the sterilizing device 10 and control the operation of the sterilizing device 10 depending on a position on the steering wheel gripped by the driver recognized by the grip recognition unit 20, and may control the operating time of the sterilizing device 10 depending on the time for which the driver grips the steering wheel.

Accordingly, it is possible to efficiently control the operation of the sterilizing device 10 to reduce battery consumption of the vehicle and to sterilize the steering wheel to provide comfortable driving.

The grip recognition unit 20 may be connected to the camera sensor 22 provided in the vehicle or a pressure sensor 21 mounted on the steering wheel to recognize a position on the steering wheel gripped by the driver or a time for which the driver grips the steering wheel.

As shown in FIG. 3, the grip recognition unit 20 may be connected to the camera sensor 22 provided in the vehicle and calculates a time for which the driver grips the steering wheel and a position on the steering wheel gripped by the driver. Further, the grip recognition unit 20 may be connected to a sensing device such as the pressure sensor 21 mounted on the steering wheel as well as the camera sensor 22 and calculates a position on the steering wheel gripped by the driver and a time for which the driver grips the steering wheel.

The steering wheel sterilization system may further include a driver recognition unit 50 for recognizing or detecting a driver status indicating whether a driver is riding in the vehicle or driver information of the driver, and the controller 30 may control the operation of the sterilizing device 10 on the basis of the driver status or driver information detected by the driver recognition unit 50.

As shown in FIG. 3, the driver recognition unit 50 may be connected to the camera sensor 22 provided in the vehicle and recognize whether a driver is riding in the vehicle or information on the driver.

The camera sensor 22 may capture an image of the driver riding in the vehicle to check whether the driver is riding in the vehicle and capture an image of the face or pupil of the driver to recognize information on the driver.

In addition, the driver recognition unit 50 may be connected to a sensing device such as a pressure sensor provided in a seat to check whether a driver is riding in the vehicle and may be connected to an individual recognition device such as a fingerprint sensor to recognize information on the driver.

Upon checking whether a driver is riding in the vehicle through the driver recognition unit 50, the controller 30 may learn a time when a driver is not riding in the vehicle and control the operating time of the sterilizing device 10 on the basis of the time.

In addition, based on the information on the driver recognized by the driver recognition unit 50, the controller 30 may store positions on the steering wheel gripped by the driver and times for which the driver grips the steering wheel and control the sterilizing device 10 such that the sterilizing device 10 operates in the same manner thereafter to improve the operation efficiency of the sterilizing device 10.

The controller 30 may control the sterilizing device 10 such that the sterilizing device 10 is activated to operate when the driver recognition unit 50 recognizes that the driver has alighted from the vehicle.

The driver recognition unit 50 may recognize a time when a driver rides in the vehicle or alights from the vehicle, and the controller 30 may operate the sterilizing device 10 in a state in which the driver has alighted from the vehicle on the basis of the recognized time.

Accordingly, sterilization of the steering wheel is completed when the driver rides in the vehicle, and thus a clean steering wheel can be provided to the driver.

The controller 30 may simultaneously operate a heater 60 of the vehicle when operating the sterilizing device 10.

The controller 30 may control an air conditioner provided in the vehicle when operating the sterilizing device 10 in a state in which the driver has alighted from the vehicle such that the heater 60 and the sterilizing device 10 can simultaneously operate.

Accordingly, the sterilization effect of the steering wheel can be maximized by simultaneously operating the sterilizing device 10 and the heater 60. Further, since the heater 60 is operated in a state in which the driver has alighted from the vehicle, it is not necessary to consider discomfort of the driver due to an abrupt operation of the heater 60.

The controller 30 may store a position on the steering wheel gripped by the driver or a time for which the driver grips the steering wheel, recognized by the grip recognition unit 20, according to the information on the driver recognized by the driver recognition unit 50 and control the sterilizing device 10 such that the sterilizing device 10 can immediately operate depending on the position on the steering wheel gripped by the driver or a time for which the driver grips the steering wheel according to the information on the driver.

The driver recognition unit 50 may recognize information on drivers of the vehicle, and the controller 30 may store a position on the steering wheel gripped by each driver or a time for which each driver grips the steering wheel, and when a driver rides in the vehicle, control the operating range and operating time of the sterilizing device 10 on the basis of the prestored position on the steering wheel gripped by the driver or time for which the driver grips the steering wheel.

Accordingly, it is possible to efficiently control the operation of the sterilizing device 10.

The driver recognition unit 50 may be connected to the camera sensor 22 provided in the vehicle and recognize whether a driver is riding in the vehicle or information on the driver.

As shown in FIG. 3, the driver recognition unit 50 may recognize whether a driver is riding in the vehicle or information on the driver through the camera sensor provided in the vehicle.

Accordingly, it is possible to simultaneously check whether a driver is riding in the vehicle and information on the driver through one camera sensor 22, thereby reducing cost.

The controller 30 may control the operating range of the sterilizing device 10 depending on a position on the steering wheel gripped by the driver recognized by the grip recognition unit 20.

The sterilizing device 10 may be operated by dividing the range of the steering wheel, the grip recognition unit 20 may recognize a range of positions on the steering wheel gripped by the driver, and the controller 30 may control the sterilizing device 10 such that the sterilizing device 10 performs sterilization focusing on the recognized range of the steering wheel.

Accordingly, it is possible to reduce a steering wheel sterilization time and improve battery usage efficiency.

The controller 30 may control the intensity of operation of the sterilizing device 10 depending on a time for which the driver grips the steering wheel recognized by the grip recognition unit 20.

The grip recognition unit 20 may calculate a time for which the driver grips the steering wheel while riding, and the controller 30 may calculate the operating time or the intensity of operation of the sterilizing device 10 on the basis of the calculated time and control the operation of the sterilizing device 10.

Accordingly, it is possible to provide a comfortable driving environment to the driver by perfectly sterilizing the steering wheel.

The steering wheel sterilization system may further include a signal generator 40 for generating a steering wheel sterilization request signal depending on the position on the steering wheel gripped by the driver or a time for which the driver grips the steering wheel, recognized by the grip recognition unit 20.

When a gripping time, preset according to the position on the steering wheel gripped by the driver or a time for which the driver grips the steering wheel calculated by the grip recognition unit 20, increases, the signal generator 40 for requesting that the driver operate the sterilizing device 10 may be operated.

Although the controller 30 may control the sterilizing device 10 such that it operates in a state in which the driver has alighted from the vehicle, if the driver is riding in the vehicle for a long time or sterilization cannot be performed in a state in which the driver has alighted, the controller 30 may generate a signal for requesting operation of the sterilizing device 10 while the driver is riding in the vehicle through the signal generator 40.

Accordingly, the sterilizing device 10 can be operated even when the driver is riding in the vehicle, thereby sterilizing the steering wheel to provide a comfortable driving environment.

The signal generator 40 may generate a steering wheel sterilization request signal through a display device 41 or a speaker 42 provided in the vehicle.

The signal generator 40 may request operation of the sterilizing device 10 by the driver through the display device 41 or the speaker 42 provided in the vehicle, and the controller 30 may receive an approval signal from the driver and operate the sterilizing device 10.

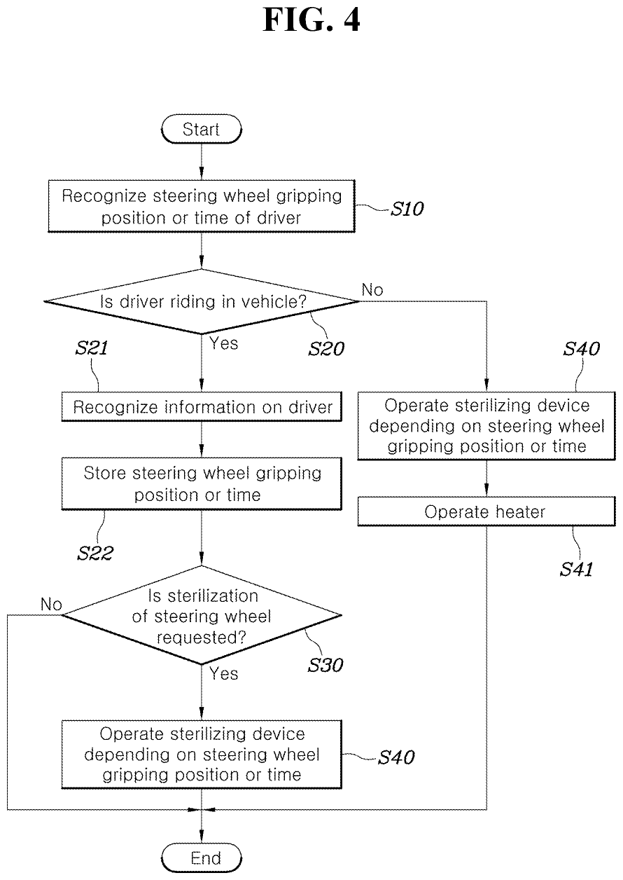

FIG. 4 is a flowchart of a method for sterilizing a steering wheel according to an embodiment of the present invention.

A preferred embodiment of a method for sterilizing a steering wheel according to the present invention will be described with reference to FIG. 4.

A method for controlling a steering wheel sterilization system according to the present invention includes a step (S10) of recognizing a position on a steering wheel gripped by a driver or a time for which the driver grips the steering wheel, and a step (S40) of controlling the operation of the sterilizing device 10 on the basis of the position on the steering wheel gripped by the driver or the time for which the driver grips the steering wheel, recognized in the recognition step.

The method may further include a step (S20) of recognizing whether or not a driver is riding in the vehicle, and in the controlling step S40, the sterilizing device 10 may be operated upon recognizing that the driver has alighted from the vehicle.

The method may further include a step (S41) of simultaneously operating the heater 60 of the vehicle when the sterilizing device 10 is operated after the driver has alighted from the vehicle.

The method may further include a step (S21) of recognizing information on the driver, and a step (S22) of storing the position on the steering wheel gripped by the driver or the time for which the driver grips the steering wheel according to the information on the driver, and in the controlling step (S40), the sterilizing device 10 may be operated depending on the stored position or time.

The method may further include a step (S30) of generating a steering wheel sterilization request signal depending on the position on the steering wheel gripped by the driver or the time for which the driver grips the steering wheel recognized in the step (S10) of recognizing a position on the steering wheel gripped by the driver or a time for which the driver grips the steering wheel, before the step (S40) of controlling the operation of the sterilizing device 10.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A steering wheel sterilization system comprising:
a sterilizing device configured to sterilize a steering wheel of a vehicle;
a grip recognition unit configured to detect (1) a grip position of a portion on the steering wheel that is gripped by a driver, and/or (2) a grip time for which the driver grips the steering wheel; and
a controller configured to control the sterilizing device to sterilize the steering wheel based on the detected grip position and/or grip time.

2. The steering wheel sterilization system of claim 1, wherein the grip recognition unit is connected to a camera sensor positioned at the vehicle and/or a pressure sensor positioned at the steering wheel.

3. The steering wheel sterilization system of claim 1, further comprising a driver recognition unit configured to detect (1) a driver status indicating whether a driver is in the vehicle, and/or (2) driver information of the driver,
wherein the controller is further configured to control the sterilizing device to selectively sterilize the steering wheel based on the detected driver status or driver information.

4. The steering wheel sterilization system of claim 3, wherein the controller is configured to control the sterilizing device to sterilize the steering wheel when the detected driver status indicates that the driver has alighted from the vehicle.

5. The steering wheel sterilization system of claim 4, wherein the controller is configured to simultaneously activate the sterilizing device and a heater of the vehicle.

6. The steering wheel sterilization system of claim 3, wherein the controller is configured to:
store, at a data storage, the detected grip position and/or grip time, wherein the stored grip position or grip time is associated with the detected driver information; and
control the sterilizing device to selectively sterilize the steering wheel based on the detected driver information and the stored grip position and/or grip time associated with the detected driver information.

7. The steering wheel sterilization system of claim 3, wherein the driver recognition unit is connected to a camera sensor that is positioned at the vehicle and configured to recognize the driver status and/or driver information.

8. The steering wheel sterilization system of claim 1, wherein the controller is configured to adjust a sterilization range based on the detected grip position.

9. The steering wheel sterilization system of claim 1, wherein the controller is configured to adjust a sterilization intensity based on the detected grip time.

10. The steering wheel sterilization system of claim 1, further comprising a signal generator configured to generate a steering wheel sterilization request signal based on the detected grip position and/or grip time.

11. The steering wheel sterilization system of claim 10, wherein the signal generator is configured to output, via a display and/or speaker of the vehicle, an indication of the steering wheel sterilization request signal.

12. A method for sterilizing a steering wheel of a vehicle, comprising:
detecting a grip position of a portion on the steering wheel gripped by a driver and/or a grip time for which the driver grips the steering wheel; and
controlling a sterilizing device to sterilize the steering wheel based on the detected grip position and/or grip time.

13. The method of claim 12, further comprising detecting a driver status indicating whether or not a driver is in the vehicle,
wherein controlling the sterilizing device comprises controlling the sterilizing device to sterilize the steering wheel when the detected driver status indicates that the driver has alighted from the vehicle.

14. The method of claim 13, further comprising activating a heater of the vehicle simultaneously with controlling the sterilizing device to sterilize the steering wheel when the detected driver status indicates that the driver has alighted from the vehicle.

15. The method of claim 12, further comprising:
detecting driver information of the driver; and
storing, at a data storage, the detected grip position and/or grip time, wherein the stored grip position or grip time is associated with the detected driver information,
wherein controlling the sterilizing device comprises controlling the sterilizing device to selectively sterilize the steering wheel based on the stored grip position and/or grip time.

16. The method of claim 12, further comprising selectively generating, based on the grip position and/or grip time, a steering wheel sterilization request signal prior to controlling the sterilizing device to sterilize the steering wheel.
